(19) United States
(12) Patent Application Publication
(10) Pub. No.: US 2004/0236377 A1
(43) Pub. Date: Nov. 25, 2004

(54) System and method for enhancing cardiac signal sensing by cardiac pacemakers through genetic treatment

(75) Inventors: Kenneth B. Stokes, Anoka, MN (US); Josee Morissette, Minneapolis, MN (US)

Correspondence Address: Kenneth J. Collier, Medtronic, Inc., 710 Medtronic Parkway N.E., Minneapolis, MN, 55432, US

(73) Assignee: Medtronic, Inc.

(21) Appl. No.: 10/685,237

(22) Filed: Oct. 14, 2003

Related U.S. Application Data
(63) Continuation of application No. 09/896,994, filed on Jul. 2, 2001, now Pat. No. 6,665,563.

(51) Int. Cl. A61N 1/362

(52) U.S. Cl. 607/3; 604/21

(57) ABSTRACT

Delivery systems for and methods of delivering ion channel protein genetic material to cardiac cells in areas adjacent to where an electrode is to be positioned in a patient's heart to improve or correct the signal to noise ratio of cardiac signals, such as the P-wave, is described herein. More specifically, there is provided a system and method for delivering sodium ion channel proteins or nucleic acid molecules encoding sodium ion channel proteins to a site in the heart adjacent to an electrode to increase the expression of the same, thereby enhancing the cardiac signal amplitude and enabling improved sensing of cardiac signals by an implanted pacemaker.

### FIELD OF THE INVENTION

The present invention relates to systems for and methods of genetically enhancing cardiac signals for use by cardiac pacemakers and, more particularly, for enhancing the signal to noise ratio of atrial P-waves for improved pacemaker sensing.

### BACKGROUND OF THE INVENTION

The cardiac pacemaker is a widely used device for treating various cardiac disorders, e.g., sick sinus syndrome, “brady-tachy syndrome” and heart block. The basic function of the pacemaker is to deliver stimulus pulses to one or more of the patient's heart chambers, as and when needed, to initiate cardiac depolarizations and thus maintain a desired heart rate, or to affect improvements in cardiac output for patients in heart failure. In addition to delivering stimulus pulses, another important feature is the sensing of a patient's heartbeat signals, when they occur spontaneously, for purposes of controlling the stimulus pulse delivery. Thus, the demand pacemaker inhibits delivery of a stimulus pulse and resets the pulse generator in the event of sensing a timely spontaneous beat, i.e., a P-wave which is an atrial depolarization, or a QRS, or just R-wave, which is a ventricular depolarization. For example, an AAI mode pacemaker both paces and senses in just the atrium, and inhibits delivery of a pace pulse if a timely P-wave is sensed. The inhibit operation necessarily depends upon reliably sensing spontaneous P-waves. In a dual chamber pacemaker, both the P-wave and R-wave are sensed. As examples of dual chamber pacemakers, see U.S. Pat. Nos. 4,920,965; 4,539,991; and 4,554,921, incorporated herein by reference. A particular purpose of the dual chamber pacemaker may be to treat a block condition, where the patient's natural pacemaker is operating normally, causing timely atrial contractions, but the depolarization signal is not efficiently propagated to the ventricle so as to cause a following ventricular contraction. In such a situation, the dual chamber pacemaker is designed to sense the P-wave, and deliver a synchronized ventricular stimulus pulse, i.e., a pulse which stimulates the ventricle after a timed AV delay which approximates the AV delay of a healthy heart. It is seen that reliable sensing of the P-wave is vital to this type of dual chamber pacing.

In yet another type of pacemaker operation, the pacemaker operates in what is referred to a VDD mode, meaning that it paces only in the ventricle, but senses both P-waves and R-waves, i.e., has single chamber pacing but dual chamber sensing. The advantage of this mode is that only one lead need be positioned in the patient's heart, since no pacing pulses are delivered to the atrium. The VDD lead has the normal electrode or electrode pair at its distal end, for positioning in the ventricle; and it has a “floating” electrode (or electrode pair) proximal to the tip and positioned so that it is located in the atrium, for sensing the P-wave. See, for example, U.S. Pat. No. 5,127,694. However, since such a floating electrode is not necessarily embedded into or positioned adjacent the myocardium, the sensed P-wave is not as strong as for the case where a separate atrial lead is used, and consequently, the reliability of sensing the P-wave is even less.

Atrial sensing is additionally considered to be a significant problem because of the low P-wave amplitudes commonly available and the presence of relatively large far field QRS and other “noise” signals. It is commonly accepted that atrial P-wave amplitudes are relatively low compared to ventricular R-waves because of the differences in muscle mass near the electrodes. That is, ventricular R-waves are large because there is a large volume of myocardium around the electrode, whereas the atrial signal is small because the underlying tissue is relatively thin. Thus, for any pacing system which senses the P wave, such as an AAI pacer or any dual sense mode pacer, reliably sensing P-waves is a major problem for which improvement has long been sought.

With regard to the source of the P-wave, it is noted that it is not the muscle itself that is sensed, but the electric potentials resulting from the depolarization of several myocardial cells, i.e., a net positive ion flow into myocardial cells through specialized membrane proteins called voltage-gated ion channels, such as the sodium channels. More muscle mass means there are more membrane channels in the area adjacent to the electrodes. However, the muscle mass adjacent to the atrial electrode cannot be increased. But the P-wave could be enhanced if the number of conducting membrane channels within the adjacent muscle mass can be increased. Sodium channels are transmembrane proteins responsible for the rapid transport of Na⁺ ions across cell membranes underlying the depolarization of the action potential in many types of cells. In particular, cardiac fast sodium channels are responsible for the fast upstroke or phase 0 of the action potential in myocardial cells. Fozzard, et al., Circ. Res., 1985, 56, 475-485. Recently, a human cardiac voltage-dependent sodium channel, hH1, has been cloned, sequenced, and functionally expressed. Gellens, et al., Proc. Natl. Acad. Sci. USA, 1992, 89, 554-558.

Gene therapy has also recently emerged as a powerful approach to treating a variety of mammalian diseases. Direct transfer of genetic material into myocardial tissue in vivo has recently been demonstrated to be an effective method of expressing a desired protein. For example, direct myocardial transfection of plasmid DNA by direct injection into the heart of rabbits and pigs (Gal, et al., Lab. Invest., 1993, 68, 18-25), as well as of rats (Acsadi, et al., The New Biol., 1991, 3, 71-81), has been shown to result in expression of particular reporter gene products. In addition, direct in vivo gene transfer into myocardial cells has also been accomplished by directly injecting adenoviral vectors into the myocardium. French, et al., Circulation, 1994, 90, 2415-2424, and PCT Publication WO 94/11506.

Pursuant to the above, this invention provides a system and method of enhancing the cardiac pacemaker atrial and/or ventricular sensing function, i.e., enhancing the signal to noise ratio of cardiac signals, and in particular the sensed P-wave, through concurrent genetic treatment whereby the number of ion channels responsible for depolarization of the atrial or ventricular myocardial cells is increased. Applicants' invention is directed to introducing ion channel protein genetic material into myocardial cells adjacent to or closest to the position of the atrial or ventricular electrode. In any particular application, the genetic material is placed so as to provide maximum benefit for sensing P-waves, or other cardiac signals, with the pacing lead used, i.e., for an AAI pacing system, a lead which is fixated against the atrial wall.

### SUMMARY OF THE INVENTION

In accordance with the above, a primary purpose of Applicants' claimed invention is to provide methods and delivery systems for enhancing cardiac pacemaker signal sensing. In a particular embodiment, the claimed invention provides methods and delivery systems for enhancing cardiac pacemaker P-wave sensing. Upon identifying a patient in which the signal to noise ratio for atrial or ventricular sensing is problematic, ion channel protein genetic material is selected such that expression of a selected ion channel protein in cells adjacent to the position of the atrial or ventricle electrode corrects or improves the signal to noise ratio for cardiac signal sensing. Preferably, expression of a selected ion channel protein can improve or correct the signal to noise ratio for cardiac signal sensing in either or both the ventricles and atria of all persons with pacemakers, especially those persons which have been diagnosed with a low signal to noise ratio for P-wave sensing. Improvement or correction of P-wave sensing can be manifested by an increase in the amplitude of the P-wave, or other characteristic of the cardiac signal, thus resulting in an increase of the signal to noise ratio of the signal sensed in the pacemaker atrial sensing channel. Delivery of the ion channel protein genetic material can be accomplished by adaptation of available pacing leads, such as, for example, AAI or DDD leads, as well as by specific modification of leads and catheters. Delivery of the genetic material may be affected by a pump or may be passive.

The ion channel protein genetic material used in the system and method of this invention comprises recombinant nucleic acid molecules comprising a nucleic acid molecule encoding the ion channel protein inserted into a delivery vehicle, such as, for example, plasmids or adenoviral vectors, and the appropriate regulatory elements. Alternatively, the ion channel protein genetic material comprises the ion channel protein itself. Expression of the desired ion channel protein from recombinant nucleic acid molecules is controlled by promoters, preferably cardiac tissue-specific promoter-enhancers, operably linked to the nucleic acid molecule encoding the ion channel protein. The conduction protein is preferably a sodium ion channel protein, such as, for example, the voltage-dependent sodium channel hH1, which is used to correct or improve the signal to noise ratio of cardiac signals, and in particular, atrial P-wave sensing. The ion channel protein genetic material is delivered to specific sites adjacent to the atrial or ventricular electrode within the heart by perfusion or injection of a therapeutically effective amount, which is that amount which corrects or improves the signal to noise ratio of the cardiac signal of the myocardial cells adjacent to the electrode. The therapeutically effective amount can be delivered to the specific site in the heart in a single dose or multiple doses, as desired.

In carrying out the treatment provided by this invention, the patient's signal to noise ratio for a particular cardiac signal, such as, for example, P-wave sensing, is first studied to determine whether such cardiac signal sensing is adequate or, rather, whether the patient presents a condition requiring adjustment, which is addressable by genetically modifying the particular cardiac signal amplitude of myocardial cells adjacent the atrial or ventricular electrode in accordance with this invention. However, in a preferred embodiment, all patients with pacemakers may receive the treatment described herein to improve the cardiac signal sensing by their pacemakers. The appropriate ion channel protein genetic material is then selected, which step includes selection of the nucleic acid molecule encoding the ion channel protein, delivery vehicle, and the appropriate regulatory elements, etc., as noted above. It is also determined what dose is indicated for treating the problematic cardiac signal to noise ratio depending upon the extent of the noise that is diagnosed, and whether follow-up treatments require implantation of an externally controllable delivery system. The determined ion channel protein genetic material is prepared, and loaded into the delivery system. The treatment is then effected by utilizing the delivery system to deliver the therapeutic dose to the patient, e.g., either injecting the material or perfusing the selected area of the heart adjacent the atrial or ventricular electrode. After this genetic treatment, the patient is paced in a standard manner, e.g., AAI pacing or dual chamber synchronous pacing which includes sensing the patient's P-waves and delivering synchronized ventricular stimulus pulses, such as in the VDD or DDD mode.

The present invention further provides a delivery system for delivering a therapeutically effective amount of a predetermined ion channel protein genetic material to an identified cardiac location adjacent the atrial or ventricular electrode, the genetic material being selected for amplifying the particular cardiac signal, such as, for example, the P-wave, from cardiac cells to which it is delivered, thus improving or correcting the cardiac signal to noise ratio received by the sensing electrode. The delivery system includes the selected genetic material contained in a reservoir, and a catheter or electrode subsystem for delivering the genetic material from the reservoir to the identified cardiac location so as to contact a plurality of cells in the proximity of the sensing electrode.

The delivery system may utilize an external reservoir for providing the genetic material, or alternately may utilize an implantable reservoir. In either embodiment, a controllable pump mechanism may be provided for transferring therapeutic doses of the genetic material from the reservoir, through a catheter or electrode, and to the selected cardiac location. The pump may be a mini or micro pump located within the delivery system. Alternatively, rather than using a pump mechanism, the ion channel protein genetic material can be passively delivered to the appropriate location adjacent the appropriate electrode. The catheter subsystem may be of a type for direct introduction into the myocardium, as with a transthoracic procedure, or, more preferably, a endocardial catheter having a distal tip portion adapted for positioning and injecting the genetic material into the myocardium from within a heart chamber. In a preferred embodiment, the catheter distal tip has a normally withdrawn helical needle, which is extendable when positioned in the vicinity of the selected site so as to be screwed into the heart. The needle is hollow and connects with the catheter lumen so as to receive the pumped genetic material; it has one or more ports located so as to effectively release the genetic material for transduction into the cardiac area adjacent the sensing electrode. In the case of an electrode subsystem, an implantable electrode is used in place of the catheter subsystem, which is able to deliver drugs, such as steroids, or other bioactive agents, such as, for example, ion channel protein genetic material. Such implantable electrodes with drug dispensing capabilities are set forth in U.S. Pat. Nos. 4,711,251, 5,458,631, 4,360,031, and 5,496,360, each of which are incorporated herein by reference. The delivery system can be used for one treatment and then removed, or can be implanted for subsequent treatments, in which latter case it is controllable by an external programmer type device. In another embodiment, the catheter or electrode subsystem may be combined with a pacing lead for sensing the patient's cardiac signals and for providing stimulus pulses.

### BRIEF DESCRIPTION OF THE DRAWINGS

FIG. 1 is a flow diagram presenting the primary steps involved in the practice of this invention, including selecting an appropriate genetic material, positioning delivery system against the heart wall, and expressing the genetic material in an appropriate dose into the determined location.

FIG. 2 is a schematic representation of a delivery system in accordance with this invention, illustrating delivery of genetic material into a patient's heart at the chosen location using a catheter subsystem.

FIG. 3 is a schematic drawing of the distal portion of a catheter which can be used for injecting a solution carrying chosen genetic material into a patient's heart.

FIG. 4 illustrates the distal end of a catheter, having a distal portion which encloses an osmotic pump.

FIG. 5A is a schematic representation of a delivery system in accordance with this invention, having a combined catheter and pacing lead, with a separate pump; FIG. 5B is another embodiment of a combined pacing lead and delivery catheter having a reservoir located at the distal end of the catheter.

### DESCRIPTION OF THE PREFERRED EMBODIMENTS

Applicants' invention provides methods and delivery systems for correcting or improving cardiac signal sensing, especially the signal to noise ratio of the atrial P-wave, thus enhancing pacemaker sensing. A problematic signal to noise ratio for P-waves results from a naturally low amplitude P-wave generated in the atrium, noise from the ventricular QRS complex, muscle noise, noise from other sources, or a combination thereof. The signal to noise ratio is determined by routine and conventional techniques known to the skilled artisan. Once the specific problem has been identified in a particular patient, e.g., in any patient with a pacemaker or who is to receive a pacemaker, ion channel protein genetic material is selected such that expression of a selected ion channel protein corrects or improves the cardiac signal amplitude, thus improving or correcting the cardiac signal to noise ratio. The ion channel protein genetic material comprises either the ion channel protein itself or recombinant nucleic acid molecules comprising a nucleic acid molecule encoding the ion channel protein inserted into a delivery vehicle, such as, for example, plasmid, cosmid, YAC vector, viral vectors, and the like, and the appropriate regulatory elements. In preferred embodiments of the present invention, the nucleic acid molecule encoding the ion channel protein is the full length coding sequence cDNA of an ion channel protein, and is inserted into a plasmid or adenoviral vector, such as, for example, pGEM3 or pBR322, and Ad5, respectively. The regulatory elements are capable of directing expression in mammalian cells, specifically human cells. The regulatory elements include a promoter and a polyadenylation signal. Expression of the desired ion channel protein is preferably controlled by cardiac tissue-specific promoter-enhancers, operably linked to the nucleic acid molecule encoding the ion channel protein. The ion channel protein is preferably a sodium channel protein, such as, for example, the hH1 voltage-regulated sodium channel, which is used to correct or improve the cardiac signal to noise ratio. The ion channel protein genetic material is preferably delivered in a pharmaceutical composition comprising, for example, the ion channel protein genetic material in a volume of phosphate-buffered saline with 5% sucrose. In some embodiments, the ion channel protein genetic material is delivered with genetic material encoding the Na⁺/K⁺ pump, which is also inserted into an appropriate delivery vehicle. The ion channel protein genetic material may also be delivered separately or in combination with class I and class IV antiarrhythmic drugs, which have been shown to increase sodium channel mRNA expression. The ion channel protein genetic material is delivered to specific sites within the heart, adjacent to the atrial or ventricular electrode, by perfusion or injection of a therapeutically effective amount, which is that amount which corrects or improves the cardiac signal to noise ratio. Preferably, the therapeutically effective amount corrects or improves the P-wave signal to noise ratio. The therapeutically effective amount can be delivered to the specific site in the heart in single or multiple doses, as desired, using the delivery systems of the invention.

The present invention also comprises a delivery system for delivering a therapeutically effective amount of ion channel protein genetic material to a specific cardiac location, adjacent the atrial or ventricular electrode, in such a way as to enhance the amplitude of the cardiac signal, thus improving or correcting the signal to noise ratio. In a first embodiment, the delivery system basically comprises a reservoir subsystem for holding the genetic material, and a catheter subsystem in communication with the reservoir subsystem for placement of the genetic material in and around the identified cardiac location. In another embodiment, the delivery system basically comprises a reservoir subsystem for holding the genetic material, and an electrode subsystem in communication with the reservoir subsystem for placement of the genetic material in and around the identified cardiac location. As seen in the following discussion of several preferred embodiments, the reservoir subsystem and catheter subsystem or electrode subsystem may be separate, or they may be combined. Preferably the reservoir contains up to 25 ml of a genetic material for delivery to the myocardium. In some applications, only a bolus of about 0.1-10 ml, or more preferably 1-5 ml, is delivered to the targeted areas. In other applications, such as where ion channel protein is being delivered in repeated doses, 25 ml or more may be used. Also, the genetic material may be diluted in a saline solution, such as, for example, phosphate-buffered saline (PBS), the reservoir holding the diluted solution for controlled delivery. Additionally, it is to be understood that the reservoir and associated control apparatus may be either implantable or external to the body, depending upon the circumstances, e.g., whether metered doses are to be administered to the patient over a period of time, or whether the delivery of the genetic material is essentially a one time treatment.

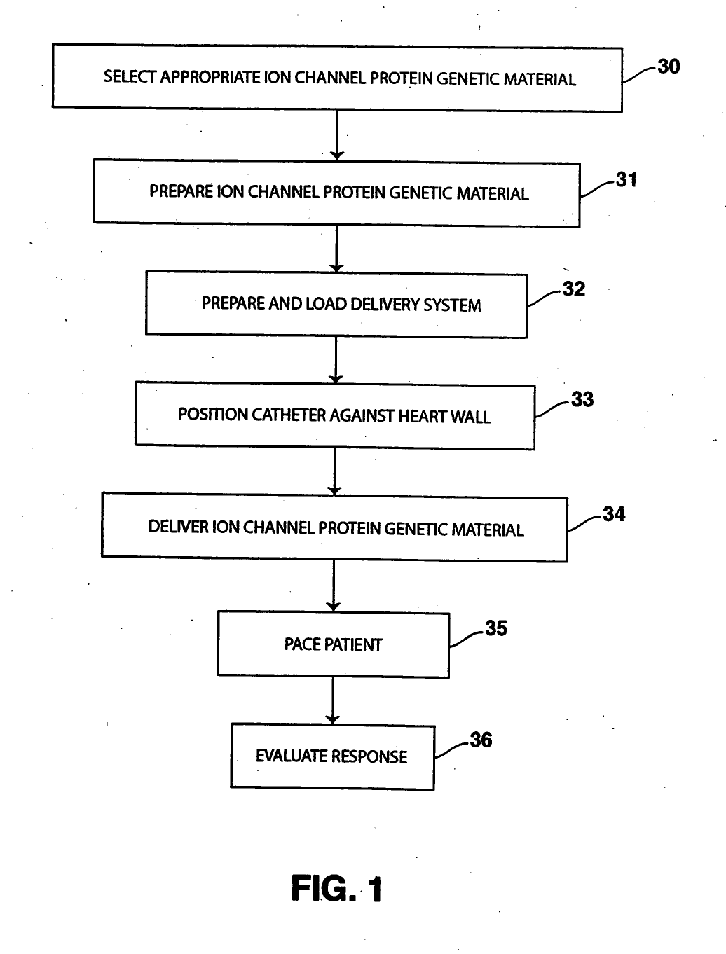

Referring now to FIG. 1, the primary steps involved in the practice of this invention are shown in the flow diagram. The illustrated steps are performed following the initial diagnosis of a patient with a problematic P-wave signal to noise ratio, which can result from a low amplitude P-wave generated in the atrium, noise from the ventricular QRS complex, noise from other sources, or a combination thereof. Diagnosis can be accomplished, for example, by electrocardiography procedures. Preferably, the steps are performed in connection with all patients having cardiac pacemakers. As illustrated in block 30, the next step is to select the appropriate ion channel protein genetic material. This selection yields the “preselected genetic material.” The ion channel protein genetic material is next prepared, as illustrated in block 31, by either inserting the nucleic acid molecules encoding the appropriate ion channel protein into a delivery vehicle with the appropriate regulatory elements, in the case of a recombinant nucleic acid molecule, or expressing the ion channel protein from an expression vector, in the case of the ion channel protein itself. As shown in block 32, the next step is to prepare and load the delivery system with a therapeutically effective amount of the ion channel protein genetic material. As illustrated in block 33, the next step comprises inserting the catheter, or other delivery subsystem, such as, for example, the electrode subsystem, into the patient's heart and positioning it against the heart wall. As shown in block 34, the next step comprises administering the therapeutically effective amount to the patient by contacting the appropriate location in the heart, adjacent to the atrial or ventricular electrode, using the delivery system described herein. An alternative method of administering the therapeutically effective amount of the ion channel protein genetic material is to directly inject the heart of the patient. The next step, shown in block 35, is to pace the patient in a standard manner, e.g., dual chamber synchronous pacing which includes sensing the patient's P-waves and delivering synchronized ventricular stimulus pulses, or AAI pacing. In accordance with this step, it may be preferable to adjust the sensitivity of the atrial or ventricular sensing channel in accordance with the observed cardiac signal amplitude. The final step 36, which is optional, is to evaluate the response of the patient to the treatment by, for example, measuring the amplitude of the cardiac signal, such as, for example, the P-wave, by conventional electrocardiographic techniques, such as, for example, by telemetry from the implanted pulse generator. The sensitivity can then be adjusted accordingly.

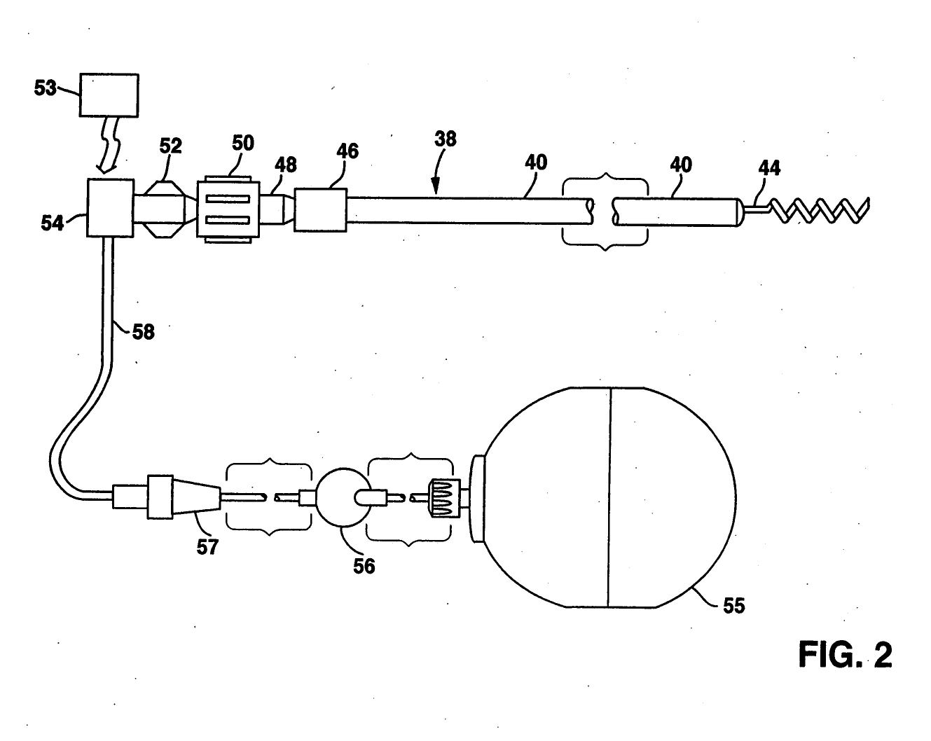

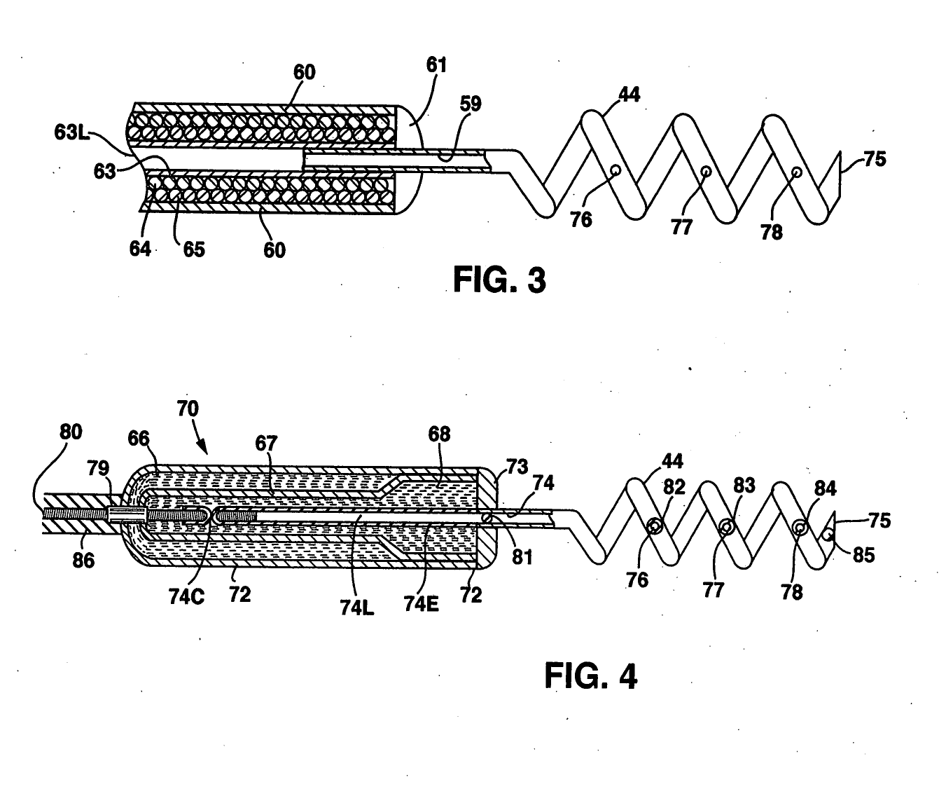

Referring now to FIG. 2, there is shown an illustrative embodiment of a delivery system useful for certain applications of this invention, e.g., where larger amounts of genetic material alone or in solution are employed. A catheter 38, preferably a transvenous catheter, includes an elongated catheter body 40, suitably an insulative outer sheath which may be made of polyurethane, Teflon, silicone, or any other acceptable biocompatible plastic. The catheter has a standard lumen (illustrated in FIG. 3) extending therethrough for the length thereof, which communicates through to a hollow helical needle element 44, which is adapted for screwing into the patient's myocardium. The outer distal end of helical element 44 is open or porous., thus permitting genetic material in fluid form to be dispensed out of the end, as is discussed in more detail below in connection with FIG. 3. At the proximal end of the catheter, a fitting 46 is located, to which a Luer lock 48 is coupled. Luer lock 48 is coupled to the proximal end of sheath 40 and receives the lumen. A swivel mount 50 is mounted to Luer lock 48, allowing rotation of the catheter relative to Luer lock 52. Luer lock 52 in turn is coupled through control element 54 to a tube 58 which communicates with reservoir 55, suitably through flow control 57 and filter 56. Reservoir 55 holds a supply of the selected genetic material. Control elements 57 and 54 are used for adjustment of the pressure and flow rate, and may be mechanically or electronically controlled. Thus, unit 54 or 57 may be used to control either rate of delivery, or dosage size, or both. Control unit 54 may be programmed to automatically release predetermined doses on a timed basis. Further, for an implanted system, control unit 54 may be activated from an external programmer as illustrated at 53. Reference is made to international application published under the PCT, International Publication No. WO 95/05781, incorporated herein by reference, for a more detailed description of such a reservoir and catheter combination. It is to be understood that such a system is useful for this invention primarily for applications where larger fluid amounts are to be expressed, e.g., where a diluted saline solution is used to wash or perfuse a selected area.

Referring now to FIG. 3, there is shown in expanded detail a schematic of the distal end of the catheter of FIG. 2, illustrating the interconnection of the helical element 44 with the interior of the catheter. As illustrated, the helical needle 44 is provided with an internal lumen 59 which is in communication with the internal lumen 63L of the lead formed by tube 63. In this embodiment, helical element 44 may also be a pacing electrode, in which case it is formed of conductive material and welded, or otherwise fastened, to tip element 61. Tip element 61 in turn is electrically connected to coil or coils 64, 65, which extend the length of the lead and are connected to a pacemaker. An outer membrane 60 forms the outer wall of elongated catheter body 40, shown in FIG. 2. Further referring to FIG. 3, element 44 has an outlet 75 where the genetic material may be expressed, and holes or ports 76, 77, and 78 may also be utilized for providing exits for the genetic material which is supplied through lumen 59 under a suitable pressure of zero up to about one atmosphere from reservoir 55 (shown in FIG. 2) and the control elements.

In practice, a catheter 38 of the form illustrated in FIGS. 2 and 3 is advanced to the desired site for treatment, eg, adjacent the site where the sensing electrode is to be positioned. The catheter may be guided to the indicated location by being passed down a steerable or guidable catheter having an accommodating lumen, for example as disclosed in U.S. Pat. No. 5,030,204; or by means of a fixed configuration guide catheter such as illustrated in U.S. Pat. No. 5,104,393. Alternately, the catheter may be advanced to the desired location within the heart by means of a deflectable stylet, as disclosed in PCT Patent Application W0 93/04724, published Mar. 18, 1993, or by a deflectable guide wire as disclosed in U.S. Pat. No. 5,060,660. In yet another embodiment, the helical element 44 may be ordinarily retracted within a sheath at the time of guiding the catheter into the patient's heart, and extended for screwing into the heart by use of a stylet. Such extensible helical arrangements are well known in the pacing art, and are commercially available.

It is to be understood that other forms of the reservoir subsystems and catheter subsystems are within the scope of this invention. Reservoir embodiments include, for example, drug dispensing irrigatable electrodes, such as those described in U.S. Pat. No. 4,360,031; electrically controllable, non-occluding, body implanting drug delivery system, such as those described in U.S. Pat. No. 5,041,107; implantable drug infusion reservoir such as those described in U.S. Pat. No. 5,176,641; medication delivery devices such as those described in U.S. Pat. No. 5,443,450; infusion pumps, such as SYNCHROMED7 made by Medtronic, Inc.; and osmotic pumps, such as those made by Alza.

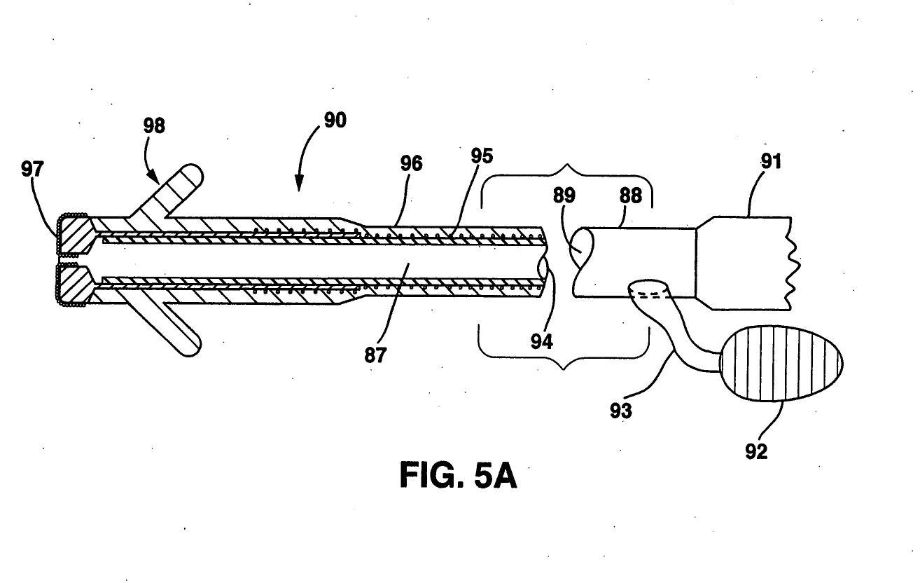

Referring now to FIG. 4, there is shown, by way of illustration, another embodiment of a delivery system having a combined catheter and reservoir, useful for applications involving delivery of a relatively small bolus of genetic material, e.g., 1-5 ml. FIG. 4 illustrates the distal end of a catheter, having a distal portion 70 which encloses an osmotic pump. See U.S. Pat. No. 4,711,251, assigned to Medtronic, Inc., incorporated herein by reference. The pump includes an inner chamber 68 and an outer chamber 66, which chambers are separated by an impermeable membrane 67. A semi-permeable outer membrane 72 forms the outer wall of chamber 66. The tubular portion 74 of the helical member connects to lumen 74L within inner chamber 68. A conductor 80, which runs the length of the catheter, extends into the inner chamber 68 and connects with extension 74E as shown at 74C to provide electrical contact through to element 44, in an application which the element 44 is used as a pacing electrode. An insulating cover 86 encompasses the conductor 80 from the point of contact with the semi-permeable outer membrane 72 distally. A seal 79 is provided at the point where the conductor passes through outer membrane 72 and inner membrane 67. An end cap 73, which may be integral with outer membrane 72 closes the chamber. Alternately, end cap 73 may be constructed to elute a predetermined medication, such as, for example, steroids. Steroids, such as dexamethasone sodium phosphate, beclamethasone, and the like, are used to control inflammatory processes.

In this arrangement, prior to inserting the catheter distal end into the patient's heart, the inner chamber 68 is charged with the genetic material which is to be dispensed into the myocardium. This may be done, for example, by simply inserting a micro needle through end cap 73, and inserting the desired bolus of genetic material into chamber 68. After the chamber 68 is filled and the is catheter is implanted, body fluids will enter chamber 66 through membrane 72 to impart a pressure on the inner chamber 68 via the impermeable membrane 67. This results in a dispensing of the genetic material stored within chamber 68 through the lumen 74L of extension 74E and through the outlet 75 of the helical element 44. Although the preferred needle or element 44 is helical, additional configurations of needles or elements can also be used as known to those skilled in the art.

Still referring now to FIG. 4, there is illustrated another embodiment of a catheter tip useful for delivering a small bolus of the selected genetic material. In this embodiment, the bolus of material is stored within the hollow interior of distal needle 44, i.e., the interior is the reservoir. The interior reservoir is maintained sealed by use of a soluble material which is normally solid, but which dissolves when subjected to body fluids for a period of time. An example of such material is mannitol. Plugs or globules 81-85 of mannitol are illustrated (by dashed lines) in place to block the two ends of element 44, as well as the ports 76, 77, 78. This may be combined with an osmotic pump, as described in connection with FIG. 3, where the outer chamber is filled with a saline solution which forces the genetic material out of the ports of element 44. Another alternate embodiment, not shown, is to use a stylet which inserted through to the distal end of the catheter, to push a piston which aids in expressing the genetic material into the myocardial cells. Alternatively, the piston can be driven by a micro pump. In another embodiment, the genetic material contacts the myocardial cells by passive delivery.

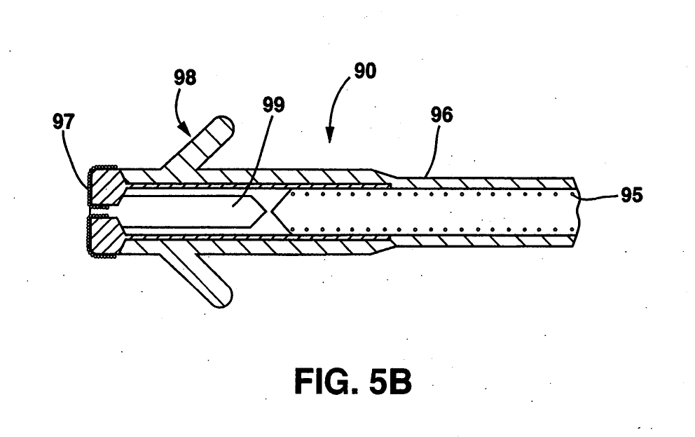

Referring now to FIG. 5A, there is shown, by way of illustration, another embodiment of an implantable delivery system comprising a combined pacing lead and delivery catheter, hereinafter referred to simply as a catheter. In this embodiment, the catheter 90 is combined with a pacemaker or pulse generator (not shown) and a source of genetic material such as illustrated by pump 92 which is suitably implanted near the pacemaker. The proximal end 91 of the catheter is connected to the pacemaker in the standard fashion. The genetic material is delivered through connecting tube 93 to a proximal section 88 of the catheter, communicating with lengthwise catheter lumen illustrated at 89. Alternately, the pacemaker head may contain a reservoir and micropump, for providing delivery of the genetic material directly to the lumen 89. The main length of the catheter has an outside sheath of biocompatible insulating material 96, and at least one conductor coil 95 which communicates electrically from the pacemaker to electrode 97 at the distal tip of the catheter. The catheter further comprises an axially positioned polymeric cannula 94, having lumen 87, through at least a portion of the catheter length and positioned within coil 95, which provides an inner surface for the catheter lumen. The cannula terminates at the distal end of the catheter, just proximal to the tip portion of electrode 97, which is illustrated as having an outer porous surface. Electrode 97 has a central opening, shown covered with the porous electrode material, through which genetic material can pass when the catheter is positioned in the patient. As shown, conductor coil 95 is electrically connected to electrode 97, and connects pace pulses and sensed cardiac signals between the pacemaker and the electrode. Of course, for a bipolar embodiment, the lead/catheter 90 carries a second electrode (not shown), suitably a ring electrode just proximal to electrode 97. Also, as illustrated, a fixation mechanism such as tines 98 are employed for fixing or anchoring the distal tip to the heart wall of the patient.

In one embodiment, pump 92 is suitably an osmotic minipump, which pumps fluid contained within through tube 93, into catheter portion 88 and through the lumens 89, 87 to the tip electrode 97. As mentioned previously, the reservoir and pump may alternately be mounted in the pacemaker device itself. In either instance, the genetic material is delivered under very minimal pressure from the reservoir through the lumen of the catheter to the electrode, where it is passed through the electrode central channel to contact myocardial cells. In yet another embodiment, the lumen portion 87 provided by the cannula is utilized as the reservoir. In this embodiment, delivery may either be passive, or with the aid of a micropump (not shown). The genetic material can be preloaded into the cannula, or it can be inserted by a needle just before the catheter is introduced and positioned with the patient.

In another embodiment, as illustrated in FIG. 5B, a chamber 99 is provided just proximal from eluting electrode 97, and serves as the reservoir of the genetic material. Insulating material 96 is formed from a self-sealing material such that it may be pierced with a needle, or the like, and reseal itself, thus allowing introduction of the genetic material into the chamber prior to implantation. Alternately, insulating material 96 can contain a port (not shown) through which the needle inserts the genetic material. In this embodiment, delivery of the material is without a pump, i.e., passive, the material draining slowly through the microporous portion of electrode 97.

The above described delivery systems can be used, for example, in methods of pacing and enhancing the detectability of sensed cardiac signals. A supply of a genetic material of the class having the property of increasing the expression of ion channels in cardiac cells to which it is delivered is selected. A transvenous catheter, having proximal and distal ends and a pacing electrode at the distal end, is introduced into the patient. The distal end of the catheter is positioned against the patient's heart wall and the genetic material is delivered through the catheter and out of the distal end, to the cardiac cells adjacent the pacing electrode, thereby enhancing cardiac signals produced by the cells. Normal cardiac pacing is carried out with the pacemaker and connected catheter implanted in the patient.

Although a transvenous form of delivery system is preferred, it is to be understood that the invention can employ other methods and devices. For example, a small bolus of selected genetic material can be loaded into a micro-syringe, e.g., a 100 μl Hamilton syringe, and applied directly from the outside of the heart.

As used herein, the phrase [symbol] cardiac signal [symbol] refers to any cardiac signal that is detectable and includes, but is not limited to, the P-wave.

As used herein, the phrase “signal to noise ratio” refers to the ratio of the amplitude of the cardiac signal, such as, for example, the P-wave, to the amplitude of the “noise.” In addition, the signal to noise ratio can be measured for other cardiac signals as well. Sources of “noise” include, but are not limited to, the QRS complex and muscle noise. It is desirable to establish a high signal to noise ratio, i.e., a signal to noise ratio of greater than 1:1 for unipolar leads and greater than 3:1 for bipolar leads. It is even more preferred to establish a signal to noise ratio greater than 10:1.

As used herein, the phrase “ion channel protein genetic material” refers to recombinant nucleic acid molecules encoding an ion channel protein or, alternatively, an ion channel protein itself, which is used in the methods and delivery systems of the invention. For chronic treatment, or long term treatment, the ion channel protein genetic material will be in the form of recombinant nucleic acid molecules encoding the ion channel protein. In contrast, for acute treatment, or short term treatment, the ion channel protein genetic material will be in the form of the ion channel proteins themselves.

A “recombinant nucleic acid molecule”, as used herein, is comprised of an isolated ion channel protein-encoding nucleotide sequence inserted into a delivery vehicle. Regulatory elements, such as the promoter and polyadenylation signal, are operably linked to the nucleotide sequence encoding the ion channel protein, whereby the protein is capable of being produced when the recombinant nucleic acid molecule is introduced into a cell.

The nucleic acid molecules encoding the ion channel proteins are prepared synthetically or, preferably, from isolated nucleic acid molecules, as described below. A nucleic acid is “isolated” when purified away from other cellular constituents, such as, for example, other cellular nucleic acids or proteins, by standard techniques known to those of ordinary skill in the art. The coding region of the nucleic acid molecule encoding the ion channel protein can encode a full length gene product or a subfragment thereof, or a novel mutated or fusion sequence. The protein coding sequence can be a sequence endogenous to the target cell, or exogenous to the target cell. The promoter, with which the coding sequence is operably associated, may or may not be one that normally is associated with the coding sequence.

The nucleic acid molecule encoding the ion channel protein is inserted into an appropriate delivery vehicle, such as, for example, an expression plasmid, cosmid, YAC vector, and the like. Almost any delivery vehicle can be used for introducing nucleic acids into the cardiovascular system, including, for example, recombinant vectors, such as one based on adenovirus serotype 5, Ad5, as set forth in French, et al., Circulation, 1994, 90, 2414-2424, which is incorporated herein by reference. An additional protocol for adenovirus-mediated gene transfer to cardiac cells is set forth in WO 94/11506, Johns, J. Clin. Invest., 1995, 96, 1152-1158, and in Barr, et al., Gene Ther., 1994, 1, 51-58, both of which are incorporated herein by reference. Other recombinant vectors include, for example, plasmid DNA vectors, such as one derived from pGEM3 or pBR322, as set forth in Acsadi, et al., The New Biol., 1991, 3, 71-81, and. Gal, et al., Lab. Invest., 1993, 68, 18-25, both of which are incorporated herein by reference, cDNA-containing liposomes, artificial viruses, nanoparticles, and the like. It is also contemplated that ion channel proteins be injected directly into the myocardium.

The regulatory elements of the recombinant nucleic acid molecules of the invention are capable of directing expression in mammalian cells, specifically human cells. The regulatory elements include a promoter and a polyadenylation signal. In addition, other elements, such as a Kozak region, may also be included in the recombinant nucleic acid molecule. Examples of polyadenylation signals useful to practice the present invention include, but are not limited to, SV40 polyadenylation signals and LTR polyadenylation signals. In particular, the SV40 polyadenylation signal which is in pCEP4 plasmid (Invitrogen, San Diego, Calif.), referred to as the SV40 polyadenylation signal, can be used.

The promoters useful in constructing the recombinant nucleic acid molecules of the invention may be constitutive or inducible. A constitutive promoter is expressed under all conditions of cell growth. Exemplary constitutive promoters include the promoters for the following genes: hypoxanthine phosphoribosyl transferase (HPRT), adenosine deaminase, pyruvate kinase, β-actin, human myosin, human hemoglobin, human muscle creatine, and others. In addition, many viral promoters function constitutively in eukaryotic cells, and include, but are not limited to, the early and late promoters of SV40, the Mouse Mammary Tumor Virus (MMTV) promoter, the long terminal repeats (LTRs) of Maloney leukemia virus, Human Immunodeficiency Virus (HIV), Cytomegalovirus (CMV) immediate early promoter, Epstein Barr Virus (EBV), Rous Sarcoma Virus (RSV), and other retroviruses, and the thymidine kinase promoter of herpes simplex virus. Other promoters are known to those of ordinary skill in the art.

Inducible promoters are expressed in the presence of an inducing agent. For example, the metallothionein promoter is induced to promote (increase) transcription in the presence of certain metal ions. Other inducible promoters are known to those of ordinary skill in the art.

Promoters and polyadenylation signals used must be functional within the cells of the mammal. In order to maximize protein production, regulatory sequences may be selected which are well suited for gene expression in the cardiac cells into which the recombinant nucleic acid molecule is administered. For example, the promoter is preferably a cardiac tissue-specific promoter-enhancer, such as, for example, cardiac isoform troponin C (cTNC) promoter. Parmacek, et al., J. Biol. Chem., 1990, 265, 15970-15976, and Parmacek, et al., Mol. Cell Biol., 1992, 12, 1967-1976. In addition, codons may be selected which are most efficiently transcribed in the cell. One having ordinary skill in the art can produce recombinant nucleic acid molecules which are functional in the cardiac cells.

Genetic material can be introduced into a cell or “contacted” by a cell by, for example, transfection or transduction procedures. Transfection refers to the acquisition by a cell of new genetic material by incorporation of added nucleic acid molecules. Transfection can occur by physical or chemical methods. Many transfection techniques are known to those of ordinary skill in the art including: calcium phosphate DNA co-precipitation; DEAE-dextran DNA transfection; electroporation; naked plasmid adsorption, and cationic liposome-mediated transfection. Transduction refers to the process of transferring nucleic acid into a cell using a DNA or RNA virus. Suitable viral vectors for use as transducing agents include, but are not limited to, retroviral vectors, adeno associated viral vectors, vaccinia viruses, and Semliki Forest virus vectors.

Treatment of cells, or contacting cells, with recombinant nucleic acid molecules can take place in vivo or ex vivo. For ex vivo treatment, cells are isolated from an animal (preferably a human), transformed (i.e., transduced or transfected in vitro) with a delivery vehicle containing a nucleic acid molecule encoding an ion channel protein, and then administered to a recipient. Procedures for removing cells from mammals are well known to those of ordinary skill in the art. In addition to cells, tissue or the whole or parts of organs may be removed, treated ex vivo and then returned to the patient. Thus, cells, tissue or organs may be cultured, bathed, perfused and the like under conditions for introducing the recombinant nucleic acid molecules of the invention into the desired cells.

For in vivo treatment, cells of an animal, preferably a mammal and most preferably a human, are transformed in vivo with a recombinant nucleic acid molecule of the invention. The in vivo treatment may involve systemic intravenous treatment with a recombinant nucleic acid molecule, local internal treatment with a recombinant nucleic acid molecule, such as by localized perfusion or topical treatment, and the like. When performing in vivo administration of the recombinant nucleic acid molecule, the preferred delivery vehicles are based on noncytopathic eukaryotic viruses in which nonessential or complementable genes have been replaced with the nucleic acid sequence of interest. Such noncytopathic viruses include retroviruses, the life cycle of which involves reverse transcription of genomic viral RNA into DNA with subsequent proviral integration into host cellular DNA. Retroviruses have recently been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M. “Gene Transfer and Expression, a Laboratory Manual”, W.H. Freeman Co., New York (1990) and Murry, E. J. e.d. “Methods in Molecular Biology”, Vol. 7, Humana Press, Inc., Clifton, N.J. (1991).

A preferred virus for contacting cells in certain applications, such as in in vivo applications, is the adeno-associated virus, a double-stranded DNA virus. The adeno-associated virus can be engineered to be replication deficient and is capable of infecting a wide range of cell types and species. It further has advantages such as heat and lipid solvent stability, high transduction frequencies in cells of diverse lineages, including hemopoietic cells, and lack of superinfection inhibition thus allowing multiple series of transductions. Recent reports indicate that the adeno-associated virus can also function in an extrachromosomal fashion.

In preferred embodiments of the present invention, the recombinant nucleic acid molecules comprising nucleic acid molecules encoding the ion channel proteins, or, in the alternative, the ion channel proteins, are delivered to cardiac cells adjacent the atrial or ventricular electrode, or both, using the delivery systems set forth above. Alternatively, the ion channel protein genetic material is delivered to the cardiac cells by direct injection.

In preferred embodiments of the present invention, the nucleic acid molecules encoding the ion channel proteins comprise the full length coding sequence cDNA of an ion channel protein. Preferably, the ion channel proteins are sodium channel proteins; more preferably, the ion channel protein is the voltage-regulated sodium channel hH1. Such a nucleic acid molecule is described in the Gellens, et al., Proc. Natl. Acad. Sci. USA, 1992, 89, 554-558, and White, et al., Mol. Pharmacol., 1991, 39, 604-608 references, both of which are incorporated herein by reference, which contain the full length amino acid sequence and cDNA sequence, respectively.

Introduction of the ion channel-encoding nucleic acid molecules or the ion channel proteins to cardiac cells adjacent the atrial or ventricular electrode will result in increased expression of sodium channels, producing a larger cardiac signal, such as, for example, P-wave, and thus, an improved or corrected signal to noise ratio. Nucleic acid molecules comprising nucleotide sequences encoding hH1 sodium channel are isolated and purified according to the methods set forth in Gellens., et al., Proc. Natl. Acad. Sci. USA, 1992, 89, 554-558, and White, et al., Mol. Pharmacol.; 1991, 39, 604-608. The nucleic acid and protein sequences of hH1 sodium channel are set forth in SEQ ID NO:1 and SEQ ID NO:2, respectively. It is contemplated that nucleic acid molecules comprising nucleotide sequences that are preferably at least 70% homologous, more preferably at least 80% homologous, and most preferably at least 90% homologous to the ion channel nucleotide sequences described in SEQ ID NO:1 can also be used.

It is understood that minor modifications of nucleotide sequence or the primary amino acid sequence may result in proteins which have substantially equivalent or enhanced activity as compared to the ion channel proteins exemplified herein. These modifications may be deliberate, as through site-directed mutagenesis, or may be accidental such as through mutations in hosts which produce the ion channel proteins. A “mutation” in a protein alters its primary structure (relative to the commonly occurring or specifically described protein) due to changes in the nucleotide sequence of the DNA which encodes it. These mutations specifically include allelic variants. Mutational changes in the primary structure of a protein can result from deletions, additions, or substitutions. A “deletion” is defined as a polypeptide in which one or more internal amino acid residues are absent as compared to the native sequence. An “addition” is defined as a polypeptide which has one or more additional internal amino acid residues as compared to the wild type protein. A “substitution” results from the replacement of one or more amino acid residues by other residues. A protein “fragment” is a polypeptide consisting of a primary amino acid sequence which is identical to a portion of the primary sequence of the protein to which the polypeptide is related.

Preferred “substitutions” are those which are conservative, i.e., wherein a residue is replaced by another of the same general type. As is well understood, naturally-occurring amino acids can be subclassified as acidic, basic, neutral and polar, or neutral and nonpolar and/or aromatic. It is generally preferred that encoded peptides differing from the native form contain substituted codons for amino acids which are from the same group as that of the amino acid replaced. Thus, in general, the basic amino acids Lys, Arg, and Histidine are interchangeable; the acidic amino acids Asp and Glu are interchangeable; the neutral polar amino acids Ser, Thr, Cys, Gln, and Asn are interchangeable; the nonpolar aliphatic acids Gly, Ala, Val, Ile, and Leu are conservative with respect to each other (but because of size, Gly and Ala are more closely related and Val, Ile and Leu are more closely related), and the aromatic amino acids Phe, Trp, and Tyr are interchangeable.

While Pro is a nonpolar neutral amino acid, it represents difficulties because of its effects on conformation, and substitutions by or for Pro are not preferred, except when the same or similar conformational results can be obtained. Polar amino acids which represent conservative changes include Ser, Thr, Gln, Asn; and to a lesser extent, Met. In addition, although classified in different categories, Ala, Gly, and Ser seem to be interchangeable, and Cys additionally fits into this group, or may be classified with the polar neutral amino acids. Some substitutions by codons for amino acids from different classes may also be useful.

Once the nucleic acid molecules encoding the ion channel proteins are isolated and purified according to the methods described above, recombinant nucleic acid molecules are prepared in which the desired ion channel nucleic acid molecule is incorporated into a delivery vehicle by methods known to those skilled in the art, as taught in, for example, Sambrook et al., Molecular Cloning: A Laboratory Manual, Second Ed. Cold Spring Harbor Press (1989). Preferred delivery vehicles include, for example, plasmids (Acsadi, et al., The New Biol., 1991, 3, 71-81, and Gal, et al., Lab. Invest., 1993, 68, 18-25, both of which are incorporated herein by reference) and adenovirus (WO 94/11506, Johns, J. Clin. Invest., 1995, 96, 1152-1158, and in Barr, et al., Gene Ther., 1994, 1, 51-58, each of which are incorporated herein by reference). The nucleic acid molecules encoding ion channel proteins, or ion channel proteins produced therefrom, are delivered to the cardiac cells adjacent to the atrial electrode by the delivery systems of the present invention. Thus, such delivery systems of the present invention are used to contact the cardiac cells adjacent the atrial electrode with recombinant nucleic acid molecules encoding an ion channel protein, or ion channel proteins.

Where the ion channel protein genetic material is in the form of ion channel proteins, such proteins can be prepared in large quantities by using various standard expression systems known to those skilled in the art. Sambrook et al., Molecular Cloning: A Laboratory Manual, Second Ed. Cold Spring Harbor Press (1989), pp. 16.1-16.55, incorporated herein by reference.

The recombinant nucleic acid molecules or ion channel proteins are preferably delivered in a pharmaceutical composition. Such pharmaceutical compositions can include, for example, the recombinant nucleic acid molecule or protein in a volume of phosphate-buffered saline with 5% sucrose. In other embodiments of the invention, the recombinant nucleic acid molecule or protein is delivered with suitable pharmaceutical carriers, such as those described in the most recent edition of Remington's Pharmaceutical Sciences, A. Osol, a standard reference text in this field. The recombinant nucleic acid molecule or protein is delivered in a therapeutically effective amount. Such amount is determined experimentally and is that amount which either improves or corrects the P-wave signal to noise ratio by enhancing the P-wave amplitude as a result of the increased expression of sodium channels in the cardiac cells adjacent the atrial or ventricular electrode. The amount of recombinant nucleic acid molecule or protein is preferably between 0.01 μg and 100 mg, more preferably between 0.1 μg and 10 mg, more preferably between 1 μg and 1 mg, and most preferably between 10 μg and 100 μg. A single therapeutically effective amount is referred to as a bolus. Where adenovirus vectors are used, the amount of recombinant nucleic acid molecule is preferably between 10⁷ plaque forming units (pfu) and 10¹⁵ pfu, more preferably between 10⁸ pfu and 10¹⁴ pfu, and most preferably between 10⁹ pfu and 10¹² pfu. A single therapeutically effective amount of ion channel protein genetic material is referred to as a bolus. In some embodiments of the present invention, the delivery of the recombinant nucleic acid molecules or proteins is combined with steroid elution, such as with dexamethasone sodium phosphate, beclamethasone, and the like, to control inflammatory processes.

In some embodiments of the invention, it may be preferred to administer, in addition to ion channel protein genetic material, delivery vehicle encoding the Na⁺/K⁺ pump. The Na⁺/K⁺ pump acts to discharge Na⁺ ions from the myocardial cells that have accumulated as a result of the introduction of the ion channel protein genetic material. This treatment can be optional, as determined by the skilled practitioner. cDNA encoding the alpha and beta subunits of the human Na⁺/K⁺ pump are set forth in Kawakami, et al., J. Biochem., 1986, 100, 389-397, and Kawakami, et al., Nuc. Acids Res., 1986, 14, 2833-2844, both of which are incorporated herein by reference. The nucleic acid and amino acid sequences for the alpha subunit are set forth in SEQ ID NO:5 and SEQ ID NO:6, respectively. The nucleic acid and amino acid sequences for the beta subunit are set forth in SEQ ID NO:7 and SEQ ID NO:8, respectively. The delivery vehicles for the pump subunits can be constructed from cDNA libraries in the same manner as set forth for hH1, except that the forward primer 5′-ATGGGGAAGGGGGTTGGACGTGAT-3′ (SEQ ID NO:9) and reverse primer 5′-ATAGTAGGTTTCCTTCTCCACCCA-3′ (SEQ ID NO:10) for the alpha subunit, and the forward primer 5′-ATGGCCCGCGGGAAAGCCAAGGAG-3′ (SEQ ID NO:11) and reverse primer 5′-GCTCTTAACTTCAATTTTTACATC-3′ (SEQ ID NO:12) for the beta subunit are used. It is understood that other primers can be used in addition to those set forth herein, as is well known to the skilled artisan. A therapeutically effective amount of the genetic material encoding the Na⁺/K⁺ pump is delivered to the myocardial cells using the delivery systems described herein. The therapeutically effective amount is determined by the practitioner, and depends upon the results achieved with the ion channel protein genetic material.

In preferred embodiments of the invention, the recombinant nucleic acid molecules encoding the ion channel proteins is delivered with class I and/or class IV antiarrhythmic drugs, such as, for example, verapamil, mexiletine, and the like, or combinations thereof. These drugs may be delivered subcutaneously, intravenously, injected in the immediate vicinity of the atrial electrode, or as determined by the skilled artisan. These drugs may be delivered by one injection, or in multiple injections. The amount of antiarrhythmic drugs depends upon the age, weight, sex, and other characteristics of the patient, and is determined empirically by the skilled artisan. Class I and/or class IV antiarrhythmic drugs have been shown to enhance sodium ion channel expression in mammals. Duff, et al., Mol. Pharmacol., 1992, 42, 570-574, and Taouis, et al., J. Clin. Invest., 1991, 88, 375-378, both of which are incorporated herein by reference.

The following examples are meant to be exemplary of the preferred embodiments of the invention and are not meant to be limiting.

### EXAMPLES

### Example 1

### Isolation and Purification of Nucleic Acid Molecule Encoding hH1

Nucleic acid molecules encoding hH1 are isolated and purified according to general methods well known to those skilled in the art, and in particular, by the method set forth in Gellens, et al., Proc. Natl. Acad. Sci. USA, 1992, 89, 554-558, incorporated herein by reference. Briefly, a size selected and random-primed adult human cardiac cDNA library constructed in λZAPII (Stratagene) is screened with cDNA probes corresponding to nucleotides 1-4385 and 5424-7076 derived from the rat muscle TTX-I isoform (rSkM2), as set forth in Kallen, et al., Neuron, 1990, 4, 233-242, incorporated herein by reference. Hybridizations are performed at 42EC for 18 hours in 50% formamide, 5×SSPE, 5× Denhardt's solution, 0.1% SDS/salmon sperm DNA, random primed ³²P-labeled probe. Filters are washed with 6×standard saline citrate, 0.1% SDS at 65EC. Plaque purified clones are rescued as pBluescript phagemids and sequenced as described in Kallen, et al., Neuron, 1990, 4, 233-242. A full-length hH1 construct is made in pBluescript by sequential ligation of S14 EcOR1-Sac II (nt +1 to +252), C75 Sac II-KpnI (nt +253 to +4377), and C92 KpnI-EcOR1 (nt +4378 to +8491) fragments and the full length insert is moved into a modified pSP64T vector, as set forth in White, et al., Mol. Pharmacol., 1991, 39, 604-608, incorporated herein by reference. Nucleotides −151 to −8 of the 5′ untranslated region are deleted from the construct using exonuclease III and mung bean nuclease, as set forth in White, et al., Mol. Pharmacol., 1991, 39, 604-608.

Alternatively, cDNA for hH1 may be prepared from fresh cardiac tissue. Briefly, total cellular RNA is isolated and purified (Chomczynsky, et al., Anal. Biochem., 1987, 162, 156-159) from heart tissue, obtained from cardiac transplantation donors, or from salvaged tissue, and selected for poly(A) RNA (Sambrook et al., Molecular Cloning: A Laboratory Manual, Second Ed. Cold Spring Harbor Press (1989), pp. 7.26-7.29). cDNA corresponding to the hH1 sodium channel protein is prepared from the poly(A) cardiac RNA by reverse transcription using a GENEAMP™ PCR kit (Perkin Elmer Cetus, Norwalk, Conn.), or the like, using random hexamers according to the manufacturer's instructions. The specific hH1 nucleic acid molecules are amplified by the polymerase chain reaction (PCR), also using the GENEAMP™ PCR kit, or the like, using forward and reverse primers specific for hH1 according to the manufacturer's instructions. For example, the forward primer for cloning hH1 is preferably 5′-ATGGCAAACTTCCTATTACCTCGG-3′ (SEQ ID NO:3), and the reverse primer is 5′-CACGATGGACTCACGGTCCCTGTC-3′ (SEQ ID NO:4). It is understood that additional primers can be used for amplification as determined by those skilled in the art. These primers may be preceded at the 5′ terminus by nucleotide sequences containing endonuclease restriction sites for easy incorporation into vectors. The specific ion channel nucleic acid molecules can also be amplified by PCR from human genomic DNA (Stratagene, San Diego, Calif.). After cutting the PCR products with the appropriate restriction endonuclease(s), the PCR products are purified by phenol:chloroform extractions, or using commercial purification kits, such as, for example, MAGIC™ Minipreps DNA Purification System (Promega, Madison, Wis.). The specific nucleotide sequence of the PCR products is determined by conventional DNA sequencing procedures, and the identity of the PCR products confirmed by comparison to the published sequences for the ion channel proteins.

### Example 2

### Insertion of Ion Channel cDNA into Delivery Vehicles

Preferably, ion channel cDNA is inserted into either plasmid or adenoviral vectors. Plasmid vectors include for example, pGEM3 or pBR322, as set forth in Acsadi, et al., The New Biol., 1991, 3, 71-81, and Gal, et al., Lab. Invest., 1993, 68, 18-25. Adenoviral vectors include for example, adenovirus serotype 5, Ad5, as set forth in French, et al., Circulation, 1994, 90, 2414-2424, and Johns, J. Clin. Invest., 1995, 96, 1152-1158.

Preferably, the primers used to amplify the ion channel nucleic acid molecules are designed with unique endonuclease restriction sites located at the 5′ terminus. In the absence of such design, polylinker arms, containing unique restriction sites, can be ligated thereto. After cutting the purified PCR products with the appropriate restriction endonuclease(s), the plasmid vector, comprising a polylinker, is also cut with the same restriction endonuclease(s), affording the ion channel nucleic acid molecule a site at which to ligate. In a similar manner, recombinant adenovirus (Gluzman, et al., in Eukaryotic Viral Vectors, Gluzman, ed., Cold Spring Harbor Press, 1982, pp.187-192, French, et al., Circulation, 1994, 90, 2414-2424, and Johns, J. Clin. Invest., 1995, 96, 1152-1158) containing ion channel cDNA molecules are prepared in accordance with standard techniques well known to those skilled in the art.

It is contemplated that variations of the above-described invention may be constructed that are consistent with the spirit of the invention.

What is claimed is:

1. A delivery system for delivering a therapeutically effective amount of a predetermined genetic material to myocardial cells of a chosen location of a patient's heart, said genetic material being selected for the function of increasing the amplitude of the patient's cardiac signal so that it can be better sensed by an electrode, comprising:
a supply of said genetic material;
reservoir means for containing said genetic material; and
delivery means for delivering said genetic material from said reservoir to said myocardial cells, thereby increasing the amplitude of the cardiac signal and improving the signal to noise ratio that can be sensed by a pacemaker.

2. The delivery system of claim 1, wherein said supply of genetic material comprises a bolus of ion channel protein genetic material selected for the function of increasing the amplitude of the cardiac signal.

3. The delivery system of claim 1, wherein said delivery means comprises a catheter with a distal end portion, and said reservoir means is located in said distal end portion.

4. The delivery system of claim 3, wherein said distal end portion comprises a hollow helical element forming an interior, and said reservoir means comprises said interior with said supply therein.

5. The delivery system of claim 1, wherein said delivery means comprises a catheter with a lumen for delivering said genetic material therethrough, said catheter having a distal tip communicating with said lumen for contacting said plurality of cells in the proximity of said electrode with said genetic material.

6. The delivery system of claim 5, wherein said distal tip is a hollow helical needle tip.

7. The delivery system of claim 5, wherein said catheter is a transvenous endocardial catheter.

8. The delivery system of claim 1, wherein said reservoir contains a supply of 0.1-10 ml of said genetic material.

9. The delivery system of claim 1, wherein said delivery means comprises a catheter with a distal portion and an end tip, and wherein said reservoir means is contained in said distal portion, and further comprising force means for forcing said genetic material from said reservoir means and out of said end tip.

10. The delivery system of claim 9, wherein said force means comprises a stylet.

11. The delivery system of claim 1, wherein said delivery system comprises a hollow helical screw-in element loaded with a bolus of said genetic material.

12. The delivery system of claim 11, wherein said element comprises ports for egress of said genetic material into said identified cardiac location when said element is screwed into said location, and further comprising soluble plugs in said ports to maintain them normally closed but which dissolve when said element is positioned within said patient's heart.

13. The delivery system of claim 1, wherein said predetermined genetic material is. DNA or RNA, and imparts chronic change in ion channel expression in said cardiac cells.

14. The delivery system of claim 1, wherein said delivery means comprises a catheter with a distal end portion, and said reservoir means is located in said distal end portion.

15. The delivery system of claim 13, wherein said DNA or RNA encodes an ion channel protein.

16. The delivery system of claim 15, wherein said ion channel protein is a sodium channel protein.

17. The delivery system of claim 16, wherein said sodium channel protein is hH1.

18. The delivery system of claim 1, wherein said predetermined genetic material is protein, and imparts acute change in sodium channel expression in said cardiac cells.

19. The delivery system of claim 18, wherein said protein is an ion channel protein.

20. The delivery system of claim 19, wherein said ion channel protein is a sodium channel protein.

21. The delivery system of claim 20, wherein said sodium channel protein is hH1.

22. An implantable delivery system for delivering doses of a therapeutically effective amount of a predetermined genetic material to myocardial cells in a chosen location of a patient's heart, comprising:
a supply of genetic material of the class having the property of increasing the expression of ion channels in the myocardial cells to which it is delivered;
a catheter, said catheter having a distal tip portion for engaging the cells of said chosen location and delivering thereto said genetic material;
reservoir means for holding said supply of genetic material and providing it to said distal tip portion of said catheter; and
delivery means for delivering a therapeutically effective amount of said genetic material from said reservoir means through said distal tip portion to said chosen location.

23. The system as described in claim 20, further comprising:
control means for controlling operation of said delivery means to deliver respective said doses.

24. The implantable delivery system of claim 23, wherein said control means comprises initiating means for initiating delivery of said genetic material, said initiating means comprising an external programmer.

25. The implantable delivery system of claim 23, wherein said control means comprises automatic means for automatically initiating delivery of said genetic material.

26. An implantable delivery system for delivering predetermined genetic material to cardiac cells adjacent to a pacing electrode positioned against the inner wall of a patient's heart, comprising:
a supply of genetic material of the class having the property of increasing the expression of ion channels in cardiac cells to which it is delivered;
a catheter, said catheter having a distal tip portion for engaging said cardiac cells and delivering thereto said genetic material;
reservoir means for holding said supply of genetic material and providing it to said distal tip portion of said catheter; and
delivery means for delivering a therapeutically effective amount of said genetic material from said reservoir means through said distal tip portion to said cardiac cells.

27. The implantable delivery system of claim 26, wherein the distal end of said distal tip portion further comprises a pacing electrode.

28. The system as described in claim 26, further comprising:
control means for controlling operation of: said delivery means to deliver respective said doses.

29. The implantable delivery system of claim 26, wherein said control means comprises initiating means for initiating delivery of said genetic material, said initiating means comprising an external programmer.

30. The implantable delivery system of claim 26, wherein said control means comprises automatic means for automatically initiating delivery of said genetic material.

31. An implantable system for pacing a patient's heart and for delivering a predetermined genetic material to cardiac cells adjacent to a pacing electrode positioned in said patient's heart, comprising:
a supply of genetic material of the class having the property of increasing the expression of ion channels in cardiac cells to which it is delivered;
a catheter, said catheter having proximal and distal ends, a lumen through at least a part thereof and connecting to said distal end, a pacing electrode positioned at said distal end for engaging said patient's heart wall, said electrode having a channel therethrough in communication with said lumen, and a conductor connecting said proximal end to said electrode,
a pulse generator connected electrically to said conductor at said catheter proximal end for delivering pace pulses to said electrode,
reservoir means for holding said supply of genetic material, and
delivery means for delivering said genetic material from said reservoir to said lumen, whereby said material passes through said lumen and said channel to said heart wall.

32. The implantable system of claim 31, wherein said reservoir is mounted in said pulse generator.

33. The implantable system of claim 31, wherein said delivery means is passive.

34. The implantable system of claim 31, wherein said delivery means comprises a pump.

35. The implantable system of claim 31, wherein said electrode is substantially concentric with respect to the catheter axis, and the channel passes through the center of said electrode.
